(19) United States
(12) Patent Application Publication
(10) Pub. No.: US 2026/0049982 A1
(43) Pub. Date: Feb. 19, 2026

(54) Improving Signal to Noise in Immunoassays

(71) Applicant: Siemens Healthcare Diagnostics Inc., Tarrytown, NY (US)

(72) Inventors: Nivedita Mitra, Bangalore (IN); Ramya V M, Bangalore (IN); Mohiudeen Azhar, Bangalore (IN); Qingping Jiang, East Walpole, MA (US); Ishita Chakraborty, Bangalore (IN); David Sharpe, Foxborough, MA (US)

(73) Assignee: Siemens Healthcare Diagnostics Inc., Tarrytown, NY (US)

(21) Appl. No.: 19/104,269

(22) PCT Filed: Sep. 14, 2023
(86) PCT No.: PCT/US2023/074184

§ 371 Date: Feb. 17, 2025

Related U.S. Application Data
(60) Provisional application No. 63/375,871, filed on Sep. 16, 2022.

(51) Int. Cl. G01N 33/543; G01N 21/76

(52) U.S. Cl. CPC G01N 33/54333; G01N 21/76

(57) ABSTRACT

The present disclosure includes certain steps in immunoassays, such as immunoassays based on chemiluminescence, which increase assay sensitivity as based on a variety of metrics including increasing signal to noise and decreasing limits of detection. One strategy for these enhancements involves movement, and potential sequestration, of solid phases in one triggering reagent prior to addition of a second triggering reagent for chemiluminescence.

### FIELD OF DISCLOSURE

The present disclosure is related to immunoassays and methods and systems therefor. The strategies are particularly advantageous for chemiluminescence based immunoassays such as those involving acridinium chemiluminescence.

### BACKGROUND

Immunoassays for detection of an analyte of interest in a biological sample often operate through the use of a solid support having an analyte or a binding partner for the analyte immobilized thereon. In competitive immunoassay formats, analytes of interest in the biological sample compete to bind with conjugates having a detectable label and/or binding partners. In sandwich assay formats, analytes (and typically antigens) of interest are bound to two layers of binding partners (e.g., antibodies) for the analyte, one layer of which may be immobilized and the other is conjugated to a label. Extracting this solid phase following competitive binding and measuring the amount of label attached thereto in a controlled manner may give information about the amount of analyte in the biological sample.

Measuring the amount of label is typically contingent on what the label itself is. For example, chemiluminescent assays involve conjugation of a chemiluminescent moiety to an analyte for the competitive binding. The chemiluminescence can be measured following collection of the solid phase to determine the amount of labelled conjugates that have competitively bound to the solid support. By the addition of one or more triggering reagents to a chemiluminescent sample, chemiluminescence can be induced resulting in an amount of light output correlating with the amount of chemiluminescent moieties in the sample. Acridinium based conjugates, such as acridinium esters and acridinium sulfonamides, typically rely on triggering by an alkaline peroxide. These may be added sequentially in the form of two triggering reagents: a first triggering reagent comprising an acid such as H₂O₂ and a second basic triggering reagent (such as sodium hydroxide).

These assays have intrinsic limitations associated therewith. Various factors can decrease the signal to noise resulting in assay inconsistency and less accurate results. Assays also have an intrinsic limits of detection (LOD) where analyte concentrations below a certain level cannot be differentiated from the noise associated with the assay itself.

There is a continuous need for methods and systems for immunoassays and sample detection that increase signal to noise providing more accurate and robust immunoassays.

### SUMMARY

In accordance with the foregoing objectives and others, the present disclosure provides systems and methods which provide benefit to immunoassays, e.g., methods and systems for immunoassays and sample detection that increase signal to noise providing more accurate and robust immunoassays. For example, employing the systems and methods described herein results in increased signal to noise or decreased limits of detection as compared to an otherwise identical assay protocol. This disclosure is partially based on the addition of particle migration through a chemiluminescent triggering reagent prior to application of a second chemiluminescent reagent.

Methods such as methods for use in an immunoassay or methods for initiating or inducing chemiluminescence are provided which may include: a) adding a first chemiluminescent reagent (e.g., a triggering reagent such as an acid which may include hydrogen peroxide, nitric acid, or combinations thereof) to a reaction vessel containing magnetizable particles (e.g., paramagnetic particles, superparamagnetic particles, ferromagnetic particles, ferrimagnetic particles); and b) moving the magnetizable particles to a different location in the reaction vessel through the first chemiluminescent reagent (as measured, for example, with respect to the container of a reaction vessel). For example, in embodiments, the magnetizable particles may be moved by application of a magnetic field to the reaction vessel which may result in sequestration and migration of the particles, or substantially all of the particles, in the first chemiluminescent reagent which initiates the chemiluminescence. In some embodiments, the magnetizable particles or substantially all of the magnetizable particles are sequestered in the reaction vessel during or after the addition step by applying a magnetic field to the reaction vessel. In various implementations, the magnetizable particles or substantially all of the magnetizable particles are not sequestered in the reaction vessel during the addition step (e.g., the particles become suspended in the first chemiluminescent reagent, no magnetic field is applied to the reaction vessel).

By addition of these methods and systems in a chemiluminescent immunoassay, the measurement capabilities are typically increased. In particular, the particle migration steps may occur sequentially, or prior to the promotion and induction of chemiluminescence by the addition of a second chemiluminescent reagent such as a base (e.g., an alkali hydroxide such as sodium hydroxide or potassium hydroxide). In some embodiments, the method may further include: c) separating liquid media from the magnetizable particles after moving the magnetizable particles; and/or d) adding a second chemiluminescent reagent to the separated liquid media (e.g., in another reaction vessel) or to the separated magnetizable particles. In some embodiments, the method may include: c) adding a second chemiluminescent reagent to the magnetizable particles and, optionally, liquid media present (e.g., the first chemiluminescent reagent) after moving the magnetizable particles. Leveraging the movement and sequestration capability afforded by the magnetizable particles in any reaction position such as any chemiluminescent reagent addition position or any particle movement position may be used. For example, in some embodiments, the magnetizable particles may be clustered in the reaction vessel during the second chemiluminescent reagent addition (e.g., the reaction vessel has a magnet proximal thereto to induce clustering or accumulation). In various embodiments, the reaction vessel during the second chemiluminescent reagent addition is symmetric about a major longitudinal axis (e.g., cylindrical such as a cuvette or tube) and the second chemiluminescent reagent is added at position other than the major longitudinal axis. In embodiments, this may reduce breakup of the particle cluster when the second chemiluminescent reagent is added.

Systems for inducing a chemiluminescent reaction from a chemiluminescent sample including magnetizable particles (e.g., paramagnetic particles, superparamagnetic particles, ferromagnetic particles, ferrimagnetic particles) are also provided. For example, the system may include: an array of consecutive reaction positions for the chemiluminescent sample such that the chemiluminescent sample (or media derived therefrom) can be placed in each reaction position sequentially (e.g., by movement of a reaction vessel (e.g., a cuvette, a tube) including a material such as the magnetizable particles and/or liquid media between each reaction position, by aspiration of liquid media in a first reaction vessel and deposition into a second reaction vessel); said array of consecutive reaction positions including: a first chemiluminescent reagent addition position where a first chemiluminescent reagent can be added to the chemiluminescent sample; one or more (e.g., from one to ten, from one to five, two, three, four, five, six, seven, eight, nine, ten) particle movement positions wherein a magnetic field different from a previous position (e.g., the first chemiluminescent regent addition position, the previous particle movement position) can be applied to the chemiluminescent sample to induce movement of magnetizable particles in the first chemiluminescent reagent; a second chemiluminescent reagent addition position where a second chemiluminescent reagent is added to the chemiluminescent sample (or portion thereof or derived therefrom) after the one or more particle movement positions to induce a chemiluminescent reaction from the chemiluminescent sample (or portion thereof or derived therefrom).

In embodiments, the systems of the present disclosure may place a reaction vessel and/or sample at the indicated position along the reaction progress or schema. Migration of particles through the first chemiluminescent reagent may be considered a reaction position. For example, a reaction vessel may proceed along a track where different elements such as magnetic fields (including alternating magnetic fields at successive reaction positions) may be applied to an indicated sample in that reaction vessel.

In various implementations, the array may include a liquid transfer position where a liquid chemiluminescent sample is separated from the magnetizable particles (e.g., by aspiration with a pipette) following the one or more particle movement positions; and the second chemiluminescent reagent vessel position adds the second chemiluminescent reagent to the separated liquid sample (e.g., in a different reaction vessel such as a cuvette or tube from the reaction vessel used in the first chemiluminescent position). In some embodiments, the second chemiluminescent reagent is added to the chemiluminescent sample including the magnetizable particles (e.g., in the same reaction vessel that the first chemiluminescent sample was added such as a cuvette or tube).

In embodiments, a sample such as a biological sample may be contained a reaction vessel (e.g., a cuvette, a tube) during addition of the first chemiluminescent reagent, and the reaction vessel has a magnet adjacent thereto during addition of the first chemiluminescent reagent addition position (e.g., to sequester the magnetizable particles along an internal wall of the reaction vessel). In some embodiments, moving the magnetizable particles occurs by movement of the reaction vessel (e.g., rotating by, for example, 170-190° or 180°) with respect to the magnet. In various implementations, moving the magnetizable particles or substantially all of the magnetizable particles occurs by moving the reaction vessel into a different magnetic field created by a different set of magnets. In some embodiments, moving the magnetizable particles occurs by moving a magnet in relation to the sample (e.g., as contained in a reaction vessel) and/or modulating (e.g., increasing, decreasing) the magnetic field produced from one or more magnets (e.g., alteration of electrical parameters such as voltage or current to an electromagnet) proximal to the sample.

In embodiments, the methods and systems are most beneficial when chemiluminescence is triggered and/or promoted by the addition of multiple chemiluminescent reagents. For example, acridinium chemiluminescence typically involves the addition of a first chemiluminescence reagent (e.g., an acid such as hydrogen peroxide, nitric acid or combination thereof in a solvent) which induces oxidation of the acridinium system. This reaction may be accelerated by the addition of a second chemiluminescent reagent such as a base which alters the pH of the system to an alkaline state forming alkaline peroxides sufficient to trigger and induce chemiluminescence. In various implementations, the first chemiluminescent reagent is acidic (e.g., a reagent comprising an acid such as hydrogen peroxide, nitric acid, or a combination thereof) and optionally includes a detergent (e.g., a cationic detergent such as quaternary nitrogen or phosphorus-based salts). In some embodiments, the second chemiluminescent reagent is basic (e.g., a reagent comprising abase such as an alkali hydroxide (e.g., sodium hydroxide)) and optionally comprises a detergent (e.g., a cationic detergent such as quaternary nitrogen or phosphorus-based salts).

In embodiments, the methods and systems of the present disclosure may be used in an immunoassay format for the detection of an analyte of interest. For example, the immunoassay may involve a competitive heterogeneous assay wherein chemiluminescent conjugates compete for binding sites on the solid phase magnetizable particles with analyte in a biological sample, For example, the method may further include forming the sample by mixing magnetizable particles having a molecule capable of forming a binding complex with an analyte of interest or binding partner thereof immobilized thereon with a biological sample (e.g., blood serum, urine) and with an assay reagent including chemiluminescent conjugates (e.g., acridinium compounds such as acridinium esters or acridinium sulfonamides) capable of forming a binding complex with the molecule immobilized on the magnetizable particles; and optionally, incubating biological sample, the magnetizable particles, and the chemiluminescent conjugates; sequestering the magnetizable particles in a reaction vessel by application of a magnetic field to the reaction vessel; adding a wash buffer (e.g., a buffer solution which may contain one or more salts such as sodium chloride and sodium azide, detergents such as cationic detergents, buffering agents such as phosphate, blocking agents such as bovine serum albumin (BSA) or combinations thereof) to the magnetizable particles is added to the magnetizable particles; and separating liquid media (e.g., from the biological sample, from the assay reagents, from the first chemiluminescent reagent, from the second chemiluminescent reagent, from the wash buffer, from combinations thereof) from the magnetizable particles (e.g., the sequestered magnetizable particles).

In various implementations, the wash buffer is added when the magnetizable particles are dispersed throughout the liquid media. In some embodiments, the wash buffer is added when the magnetizable particles are sequestered (e.g., through application of a magnetic field to a reaction vessel). For example, the method may comprise at least two wash buffer additions including a first wash buffer addition where the magnetizable particles are dispersed throughout the liquid media as the wash buffer is added; and a second wash buffer addition where the magnetizable particles are sequestered during addition of the wash buffer. In some embodiments, the method further includes measuring the chemiluminescent light output following addition of the second chemiluminescent reagent (e.g., with a photomultiplier tube such as a luminometer).

### BRIEF DESCRIPTION OF FIGURES

FIG. 1 (FIGS. 1A and 1B) illustrate the progression of reaction progressions involving sequestration of magnetizable particles following addition of assay reagents to a biological sample (FIG. 1A) and aspiration of liquid media and addition of a reagent to those magnetizable particles (e.g., FIG. 1B). The top image in each individual step is a view of a reaction vessel from the side and the bottom image is a view of the reaction vessel from the top.

FIG. 2 provides exemplary particle migration induced by rotation of a reaction vessel.

FIG. 3 illustrates an exemplary particle migration involving application the use of a second magnet.

FIG. 4 provides a schematic for a particle movement involving multiple magnets.

FIG. 5 (FIGS. 5A, 5B, and 5C) illustrate reaction positions associated with addition of the second chemiluminescent reagent. The addition of the second chemiluminescent reagent (e.g., the rightmost panel in each image) may occur proximal to a PMT (e.g., in a luminometer) for subsequent measurement of chemiluminescence.

FIG. 6 is a flow chart illustrating the steps of an exemplary immunoassay of the present disclosure.

FIG. 7 (FIGS. 7A and 7B) provide elevation views of arrays (or sample holders) and a sequence of reaction vessels passing therethrough in linear (FIG. 8A) and ring (FIG. 8B) formats.

FIG. 8 compares the measurements of three assay protocols at two different analyte concentrations.

FIG. 9 (FIGS. 9A and 98) shows that particle migration allows for detection below the limit of detection of the standard assay taken at 0, 0.003, and 0.006 ng/ML. FIG. 9A compares measured concentrations using an immunoassay of the present disclosure illustrating that below the limit of detection of the standard assay (0.006 ng/mL), measurement resolution can be achieved and fully separated from the noise (0 ng/mL). FIG. 9B shows the same measurement using the standard assay protocol below its limit of detection highlighting that statistical separation cannot be achieved between measurements with noise when run on the standard assay.

FIG. 10 (FIGS. 10A and 10B) shows that particle migration allows for detection below the limit of detection of the standard assay taken at 0 and 0.004 ng/mL. FIG. 10A compares measured concentrations using an immunoassay of the present disclosure illustrating that below the limit of detection of the standard assay (0.006 ng/mL), measurement resolution can be achieved and fully separated from the noise (0 ng/mL). FIG. 10B shows the same measurement using the standard assay protocol below its limit of detection highlighting that statistical separation cannot be achieved between measurements with noise when run on the standard assay.

FIG. 11 depicts an exemplary block diagram of a computer system 1100 suitable for executing methods of the present disclosure on a chemical analyzer.

### DETAILED DESCRIPTION

Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive.

All terms used herein are intended to have their ordinary meaning in the art unless otherwise provided. All concentrations are in terms of percentage by weight of the specified component relative to the entire weight of the topical composition, unless otherwise defined.

As used herein, “a” or “an” shall mean one or more. As used herein when used in conjunction with the word “comprising,” the words “a” or “an” mean one or more than one. As used herein “another” means at least a second or more.

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

The use of ordinal number terminology (i.e., “first,” “second,” “third,” “fourth,” etc.) is solely for the purpose of differentiating between two or more items and, unless explicitly stated otherwise, is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

The term “antibody” is used herein in the broadest sense and refers to, for example, intact monoclonal antibodies and polyclonal antibodies, multi-specific antibodies (e.g., bispecific antibodies), antibody fragments and conjugates thereof that exhibit the desired biological activity of analyte binding (such as, but not limited to, Fab, Fab′, F(ab′)2, Fv, scFv, Fd, diabodies, single-chain antibodies, and other antibody fragments and conjugates thereof that retain at least a portion of the variable region of an intact antibody), antibody substitute proteins or peptides (i.e., engineered binding proteins/peptides), and combinations or derivatives thereof. The antibody can be of any type or class (e.g., IgG, IgE, IgM, IgD, and IgA) or sub-class (e.g., IgG1, IgG2, IgG3, IgG4, IgA1, and IgA2).

Detection agent: The term “detection agent” as used herein refers to any element, molecule, functional group, compound, fragment or moiety that is detectable. In some embodiments, a detection agent is provided or utilized alone. In some embodiments, a detection agent is provided and/or utilized in association with (e.g., joined to) another agent. Examples of detection agents include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes, chemiluminescent agents (such as, for example, acridinium esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes, colorimetric labels (such as, for example, dyes, colloidal gold, and the like), biotin, dioxigenin, haptens, and proteins for which antisera or monoclonal antibodies are available.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps. For example, a process, method, article, or apparatus that comprises a list of elements is not necessarily limited to only those elements but may include other elements not expressly listed or inherently present therein.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrase “associated with” includes both direct association of two moieties to one another as well as indirect association of two moieties to one another. Non-limiting examples of associations include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety.

The term “biological fluid sample” as used herein will be understood to include any liquid test sample that may be obtained from a patient and utilized in accordance with the present disclosure. Examples of biological fluid samples that may be utilized include, but are not limited to, whole blood or any portion thereof (i.e., plasma or serum), serum, EDTA plasma, lithium heparin plasma, combinations thereof, and the like.

As used herein, the term “volume” as it relates to the liquid test samples utilized in accordance with the present disclosure typically refers to a volume of liquid test sample in a range of from about 0.1 μl to about 100 μl, or a range of from about 1 μl to about 75 μl, or a range of from about 2 μl to about 60 μl, or a value less than or equal to about 50 μl, or the like.

The term “specific binding partner,” as used in particular (but not by way of limitation) herein in the term “target analyte-specific binding partner,” will be understood to refer to any molecule capable of specifically associating with the target analyte. For example, but not by way of limitation, the binding partner may be an antibody, a receptor, a ligand, aptamers, molecular imprinted polymers (i.e., inorganic matrices), combinations or derivatives thereof, as well as any other molecules capable of specific binding to the target analyte.

The term “immunoassay” as utilized herein refers to an assay to determine the presence of an analyte in a biological sample by reacting the sample with an antibody (or fragment thereof) that specifically binds to the analyte, wherein the reaction is carried out for a time and under conditions that allow for the formation of an immunocomplex between the antibody (or fragment thereof) and the analyte. In embodiments, the quantitative determination of such an immunocomplex may then be performed.

As used herein, all ranges of numeric values include the endpoints and all possible values disclosed between the disclosed values. The exact values of all half integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, from 0.1% to 2.5%, etc. It will be understood that the sum of all weight % of individual components will not exceed 100%.

By “consist essentially,” within the context of a composition, it is meant that the ingredients include only the listed components along with the normal impurities present in commercial materials and with any other additives present at levels which do not affect the operation of the embodiments disclosed herein, for instance at levels less than 5% by weight or less than 1% or even 0.5% by weight. Compositions comprising the indicated components may consist predominantly (e.g., the indicated components have the highest weight percentage in the composition such as more than 40% by weight, more than 50% by weight, more than 60% by weight, more than 70% by weight, more than 80% by weight, more than 90% by weight), consist essentially, and consist of the indicated components.

The present disclosure is based, in part, on the addition of steps to an immunoassay protocol which may result in enhancement of those assays. Typically, these steps involve the migration (and possible sequestration or re-sequestration) of magnetizable particles through a first chemiluminescent reagent such as a triggering reagent (e.g., an acidic reagent comprising H₂O₂) prior to addition of a second chemiluminescent reagent (e.g., a basic reagent such as an alkali hydroxide in a solvent).

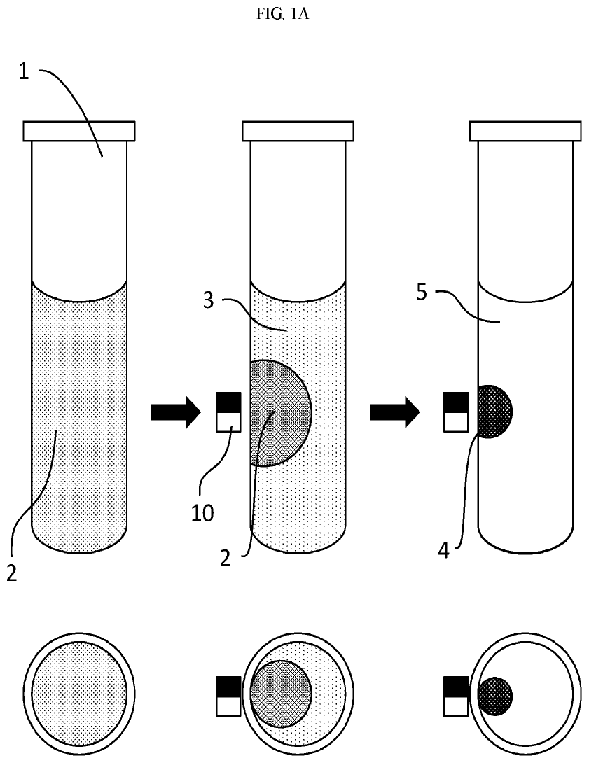

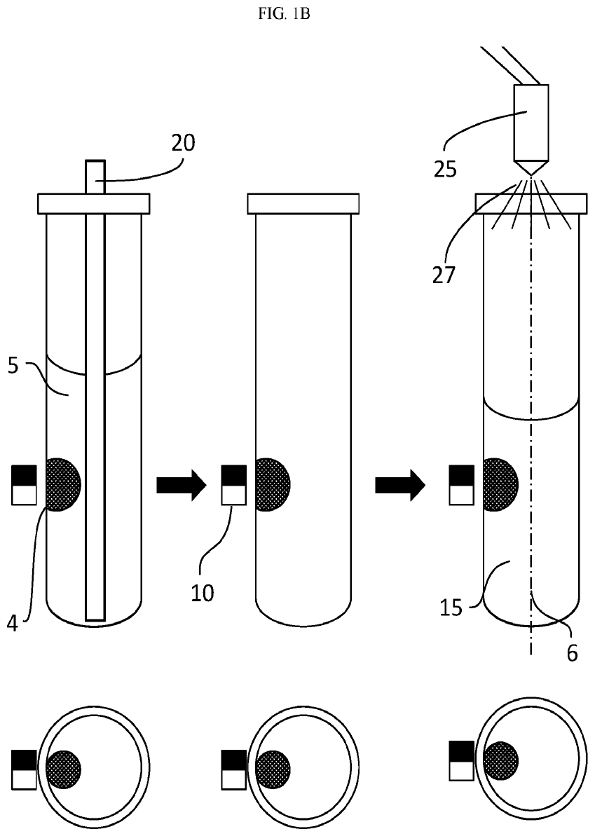

Referring now to FIG. 1 (FIGS. 1A and 1B) magnetizable particles having a molecule capable of forming a binding complex with an analyte of interest or binding partner thereof immobilized thereon, an example of the sequestering of magnetizable particles can be seen illustrating a view of the vessel from the side and a view of the vessel from the top in three stages (or reaction positions in a system). FIG. 1A shows the progression of succession from incubation of sample and reagents freely dispersed (left), migration of magnetizable particles in a magnetic field (middle), and sequestration (right). Reaction vessel 1 includes a chemiluminescent sample 2 formed by mixing a biological sample such as biological fluid sample such as blood, serum, saliva, or urine with magnetizable particles having the analyte or the binding partner for an analyte of interest in the biological sample immobilized thereon and one or more detection agents such as chemiluminescent compounds including acridinium compounds such as acridinium esters or acridinium sulfonamides capable of forming a complex with the immobilized binding partner. The magnetizable particles may be suspended in a freely distributed state in liquid media in 2, the liquid media comprising liquid components of the biological sample and assay reagents (e.g., the solvent for the chemiluminescent compounds). In the depicted embodiment, the acridinium compounds and the analyte in the sample competitively bind with the binding partners on the particles for further analysis in the assay. In some embodiments, chemiluminescent sample 2 has been further processed with one or more washes with a wash buffer as shown U.S. Pat. No. 6,143,578, which is hereby incorporated by reference in its entirety, and particularly in relation to reaction positions involving sample preparation, wash, and resuspension wash positions. In some embodiments, the methods and systems of the present disclosure may employ a resuspension wash may involving the aspiration of a liquid phase containing the unbound components of the assay, while the bound components are held in place by magnets and followed by reintroduction of wash solution into the reaction vessel.

Removal of a liquid phase may result in a portion of the liquid phase in any reaction vessel or at any reaction position being left in the reaction vessel. For example, if a reaction vessel is aspirated to remove a liquid phase, some portions of the liquid phase may be maintained in the reaction vessel as is understood with aspiration. In various implementations, aspiration may occur without taking further steps to dry any indicated reaction position. In various implementations more than 70% or more than 80% or more than 90% or more than 95% or more than 99% of the liquid phase may be removed during removal of the liquid phase (e.g., aspiration).

As can be seen in FIG. 1A, following binding and/or washing, reaction vessel 1 may be exposed to a magnetic field created by magnet 10 causing the magnetizable particles to accumulate for subsequent preparation of a chemiluminescent sample to use for chemiluminescence initiation (and light detection). In the embodiment depicted, the magnetizable particles are attracted to magnet 10 such that an increase concentration of the magnetizable particles 2 forms near magnet 10 as compared to the rest of the media 3 forms. Ultimately, the particles become sequestered 4 after exposure to magnet 10 through an appropriate time period separating from the liquid media 5.

Sequestration does not require sequestration of all magnetizable particles in the sample. For example, sequestration may involve accumulation of more than 60% or more than 70% or more than 80% or more than 90% or more than 95% or more than 99% of the magnetizable particles in the sample. Following sequestration, the liquid media 5 may be removed from the reaction vessel 1 through aspirator 20 (e.g., a tube, a pipette) leaving magnetizable particles 4 sequestered near magnet 10 in reaction vessel 1. These magnetizable particles may be considered a chemiluminescent sample used for subsequent initiation of a chemiluminescent reaction and final measurements of chemiluminescence. In the rightmost reaction position illustrated in FIG. 1B, nozzle 25 is oriented over reaction vessel 1 containing sequestered magnetizable particles (or chemiluminescent sample). Nozzle 25 is in fluid communication with a first chemiluminescent reagent (e.g., a triggering agent comprising H₂O₂, nitric acid, or a combination thereof and a solvent) reservoir and adds chemiluminescent reagent to the reaction vessel by spray 27 forming a deposit 15 of the first chemiluminescent reagent in the reaction vessel. Nozzle 25 is positioned for center addition of the first chemiluminescent reagent 27 by dispersing over the major longitudinal axis 6 of reaction vessel 1. Deposit 15 may begin to react with chemiluminescent compounds immobilized on magnetizable particles 4 to begin the process of a chemiluminescent reaction. For example, when the chemiluminescent particles are acridinium compounds, the first chemiluminescent reagent may comprise an acid (e.g., hydrogen peroxide, nitric acid, combinations thereof) and begin the oxidation reaction in the chemiluminescence reaction scheme. In various implementations, the first chemiluminescent reagent may cleave or free some chemiluminescent compounds immobilized on the particles.

Migration or movement of the particles through deposit 15 of the first chemiluminescent reagent has been found to provide enhancements of the immunoassays including systems and methods thereof described herein. For example, particle migration through the first chemiluminescent reagent is shown herein to increase the signal to noise of chemiluminescent output (e.g., as compared to an otherwise identical method or system not involving particle migration). Without wishing to be bound by theory, migration through the first chemiluminescent reagent may shorten particle exposure to the reagent (e.g., acid) and/or reduce the amount of non-specific binding released from the beads. In some embodiments, the signal to noise may be improved by more than 1.2 (e.g., more than 1.3, more than 1.4, from 1.1-5, from 1.1-4.5) as compared to an otherwise identical assay without any bead migration in the first chemiluminescent reagent.

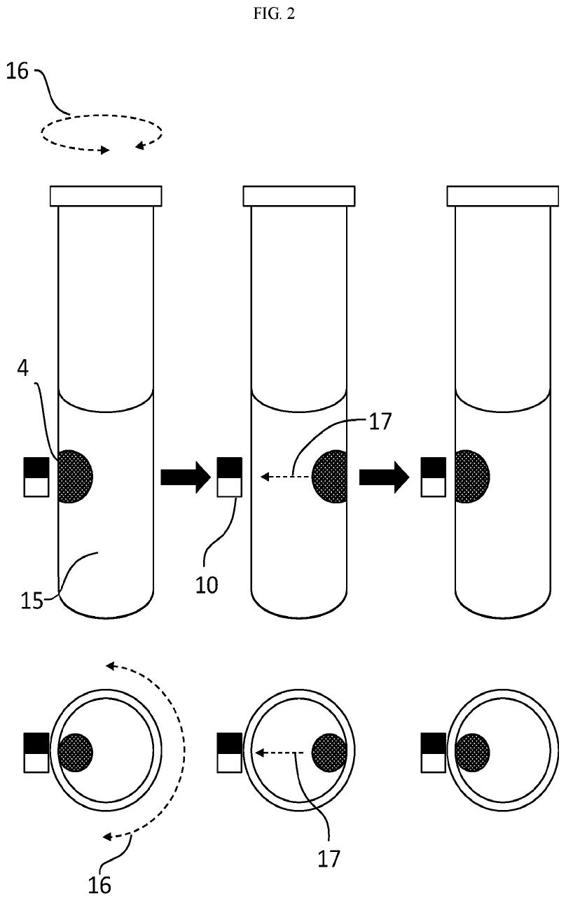

FIG. 2 provides exemplary steps to induce particle migration. Following preparation of sequestered particles 4 in the first chemiluminescent reagent 15 (e.g., an triggering reagent such as acid reagent including hydrogen peroxide), the reaction vessel may be moved within the magnetic field such that the magnetizable particles are subjected to different forces (e.g., magnetic forces, normal forces). For example, the reaction vessel may be rotated 16 by, for example 180°. This movement induces a shift in the magnetic field and normal forces experienced by the magnetizable particles. By rotating the reaction vessel and exposing the particles to different forces, the particles move through the first chemiluminescent reagent as indicated by arrow 17 resulting in re-accumulation in the reaction vessel proximal to the magnet.

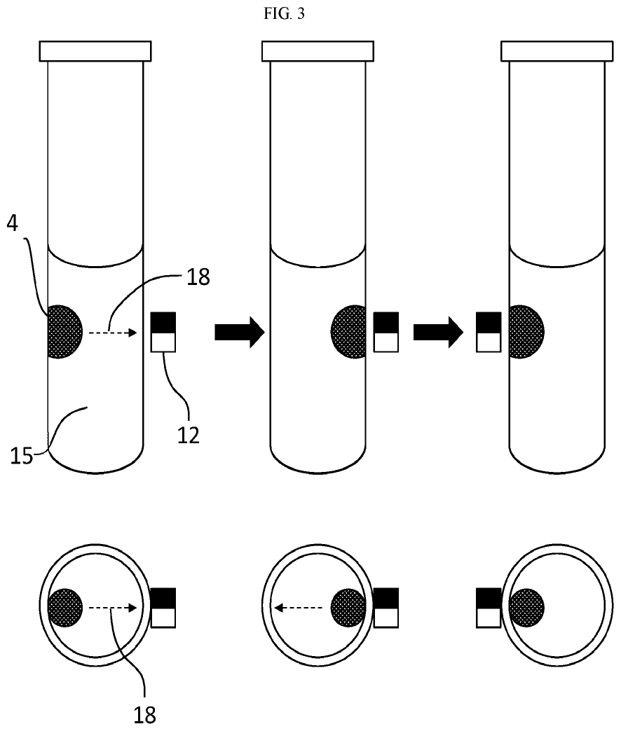

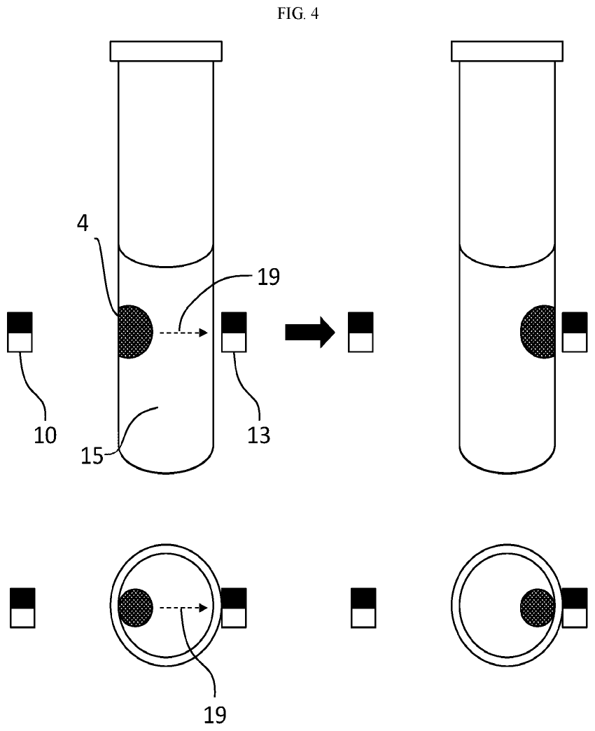

Any reaction position or step involving exposing the magnetizable particles to an altered magnetic field (as compared to an immediately preceding step or reaction position) to promote migration through the first chemiluminescent reagent may be used. For example, in some embodiments, the systems and methods may involve exposing a reaction vessel to a different magnet with, for example, a different magnetic field produced from a different magnet (e.g., an electromagnet) or a different positioning of one or more magnets relative to the reaction vessel and/or magnetizable particles (e.g., sequestered magnetizable particles sequestered from the previous step, freely dispersed magnetizable particles from a previous step). In some embodiments, the systems and methods may induce migration by exposing the reaction vessel to two or more magnets (e.g., electromagnets). The magnetic field may, for example, be altered by moving two or more (e.g., from two to four) magnets independently with respect to the reaction vessel and/or altering the magnetic field by application of different electrical properties (e.g., current, voltage) to any electromagnet used. Referring now to FIG. 3, a possible particle movement position following the final reaction position of FIG. 1B or 2 which involve sequestration from magnet 10. In the particle migration reaction position depicted, magnet 10 is moved away from the reaction vessel decreasing the magnetic field contribution from this magnet. A second magnet 12 is brought proximal to the opposite edge of the reaction vessel. The alteration in magnetic field induced by this change causes the previously sequestered particles to move toward magnet 12, located on the opposite side of the reaction (e.g., opposite as measured in a plane perpendicular to the longitudinal axis) indicated by arrow 18. In some embodiments, this process is repeated one or more times, wherein in each subsequent particle migration position, one magnet is brought closer (and/or field strength on the reaction vessel is increased), and a second magnet is moved further (and/or field strength on the reaction vessel is decreased). In some embodiments, the magnets are on opposite sides. In various implementations, field strengths of two or more magnets are independently altered, wherein the magnets are distributed around the periphery of the reaction vessel.

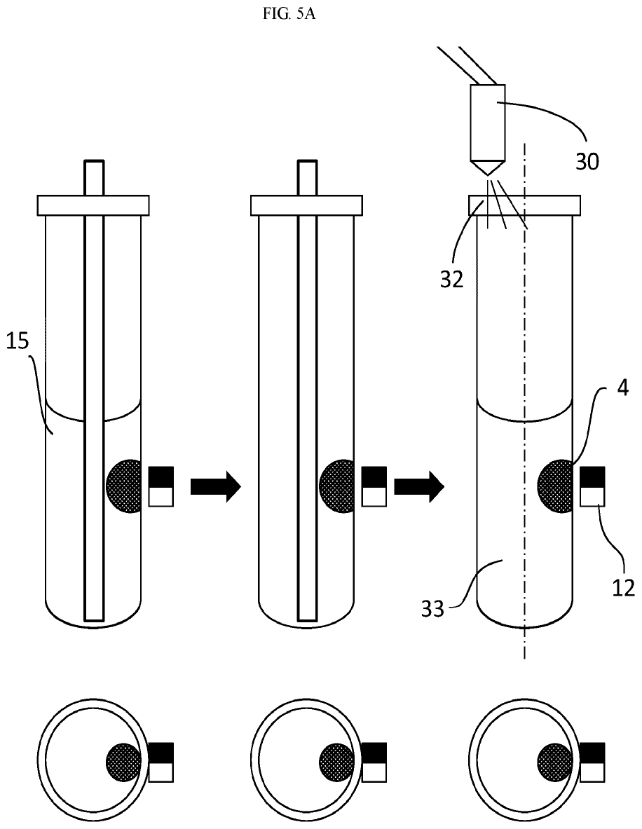

As shown in FIG. 4, accumulated particles 4 may have previously accumulated in this position due to interaction with a magnetic field (e.g., magnet 10, magnet 12). The reaction vessel may then enter a reaction position where the magnetizable particles are exposed to a second magnet 13 positioned in a manner to induce movement as indicated by arrow 19 through the first chemiluminescent reagent. Magnets 10 and 13 may be brought closer or moved away from the reaction vessel in order to control particle movements through the first chemiluminescent reagent. In some embodiments, magnets 10 and 13 are, for example, at fixed positions and the magnetic field from each magnet is independently controlled by the alteration of electrical properties thereto (e.g., current, voltage) to induce particle migration through the liquid media. Migration steps and reaction positions (e.g., as prepared by application of different magnetic fields with respect to the reaction vessel) may occur once or multiple times prior to addition of the second chemiluminescent reagent (e.g., two or more times, three or more times, four or more times, five or more times, from 2 to 10 times). For example, as shown in FIG. 4, following interaction with magnet 13 in the final step indicated on the right, the reaction vessel and magnetizable particles may be migrated a second time through the liquid media 15 by exposure to magnetic fields from magnet 10 in a subsequent step (e.g., by bringing magnet 10 closer to the reaction vessel and/or moving magnet 13 further away from the reaction vessel).

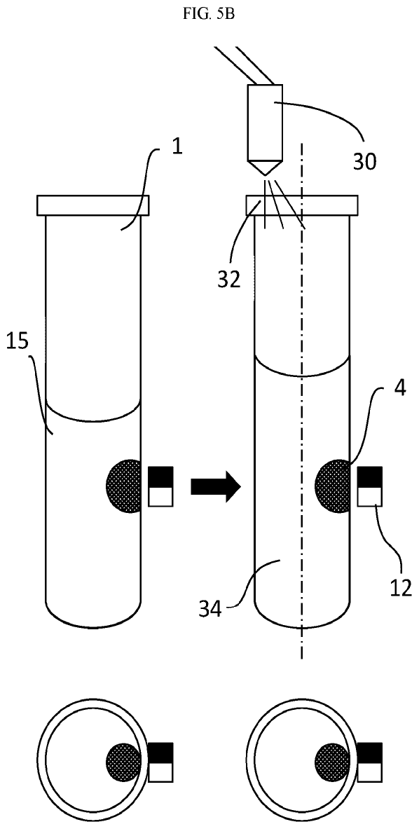

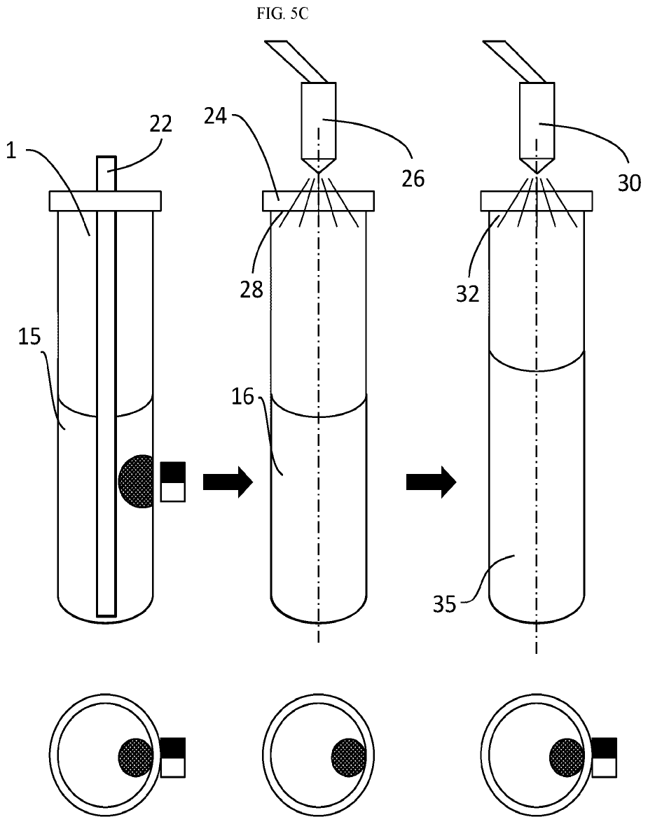

Exemplary chemiluminescence induction by the addition of the second chemiluminescent reagent may be seen in FIG. 5A. Shown on the left, magnetizable particles 4 have been migrated through first chemiluminescent reagent deposit 15 ending with sequestration proximal to magnet 12. In the embodiment depicted, first chemiluminescent reagent deposit 15 is aspirated from the reaction vessel (middle), the reaction vessel is moved into a luminometer, and the second chemiluminescent reagent 32 (e.g., a base) is deposited 33 to promote chemiluminescence from magnetizable particles 4. In some embodiments, the liquid media 33 comprises both the first chemiluminescent reagent and the second chemiluminescent reagent (e.g., the first chemiluminescent reagent is not aspirated prior to addition of the second chemiluminescent reagent, the first chemiluminescent reagent is not entirely aspirated prior to addition of the second chemiluminescent reagent). In FIG. 5B, a process is shown where no aspiration of the first chemiluminescent reagent deposit 15 occurs and the second chemiluminescent reagent 32 is added from off-center nozzle 30 directly into reaction vessel 1 to form liquid deposit 34. Liquid deposit 34 may be a combination of the first chemiluminescent reagent (e.g., an acid) the second chemiluminescent reagent (e.g., a base), and any chemiluminescent compounds (e.g., acridiniums) that have been removed from the particles during the addition of these reagents (e.g., by cleavage of an ester or sulfonamide group during the chemiluminescence process).

As can be seen in FIGS. 5A and 5B, the second chemiluminescent reagent may be added through nozzle 30 at position other than the major longitudinal axis of the reaction vessel (or off center addition). Particularly in embodiments where the final chemiluminescent sample measured includes sequestered magnetizable particles, this off-center addition of the second chemiluminescent (e.g., base) reagent may reduce deagglomeration of the sequestered particles during reagent addition as compared to center addition. This reduction in deagglomeration may increase assay consistency and accuracy.

Chemiluminescence may also be induced from a liquid deposit aspirated from the reaction vessel no comprising the magnetizable particles. Referring now to FIG. 5C, liquid deposit 15 formed from addition of the first chemiluminescent reagent to the magnetizable particles and their subsequent particle movements is used to measure the chemiluminescence and assess the concentration of analyte in the original biological sample. Liquid deposit 15 (or a portion thereof) may be aspirated from reaction vessel 1 through aspirator 22 and separated from the magnetizable particles. Subsequently, liquid deposit 15 may be added to a new reaction vessel 24 through, for example, nozzle 26 which is in operative communication with the aspirator 22 and sprays 28 into reaction vessel 24 to form liquid deposit 16 in the new reaction vessel. Liquid deposit 16 may be all of the material from liquid deposit 15 or a portion thereof (e.g., a specific volume of liquid deposit 15 such as from 1 μL to 100 mL). New reaction vessel 24 may be positioned in a luminometer prior to addition of liquid deposit 16. Nozzle 30 may then be positioned over reaction vessel 24 to spray 32 the second chemiluminescent reagent (e.g., base) onto liquid deposit 16 to induce chemiluminescence. Typically, the second chemiluminescent reagent is added to a reaction vessel in the luminometer for chemiluminescence measurement.

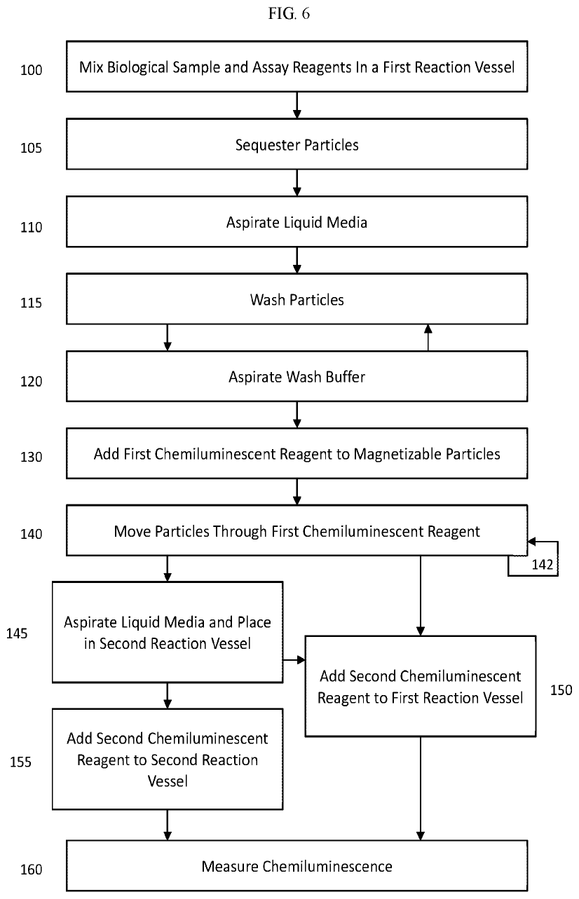

An exemplary flow chart for the process employed by embodiments of the present disclosure is shown in FIG. 6 (FIGS. 6A and 6B). Each step may be considered an independent reaction position where the systems of the present disclosure may operate to initiate the indicated reaction condition (e.g., add a reagent, sequester particles, move particles throughout an indicated reagent). Steps 100-120 involve steps for processing and reacting with a biological sample to form a chemiluminescent sample for subsequent spectroscopic analysis (e.g., as shown in FIGS. 1A and 1B). As can be seen, the particles may be washed one or more times with wash buffer.

Following preparation of the magnetizable particles, the first chemiluminescent reagent may be added at step 130. Step 140 may involve one or more particle movements as illustrated in FIGS. 2-4. These steps may involve moving one or more magnets with respect to the reaction vessel from, for example, one to five times. Following particle migration, the second chemiluminescent reagent may be added to induce (or promote) chemiluminescence. The second chemiluminescent reagent may be added, for example, to a sample in the luminometer. Particle migration may occur one or more times as indicated by arrow 142. In some embodiments, the second chemiluminescent reagent may be added to a reaction vessel comprising the magnetizable particles, having been moved through the first chemiluminescent reagent, and the chemiluminescent reagent (e.g., step 150). In some embodiments, a portion or all of the liquid media in the reaction vessel may aspirated and the second chemiluminescent reagent may be added to the first reaction vessel (e.g., step 145 followed by step 150) In some embodiments, the second chemiluminescent reagent may be added to a reaction vessel comprising the magnetizable particles following aspiration of the first chemiluminescent reagent (e.g., step 145 followed by step 155). For example, the first chemiluminescent reagent, having had the particles migrated therethrough at step 150, may be aspirated into a new reaction vessel at step 145, the new reaction vessel placed into a luminometer, the second chemiluminescent reagent added at to the first chemiluminescent reagent at step 155 and the chemiluminescence may be measured at step 160.

The magnets generally are those devices capable of creating a magnetic field which the magnetizable particles may be influenced by. For example, the magnet may be a pair of adjacent magnets of oppositely oriented polarity. The magnet may be, for example, an electromagnet.

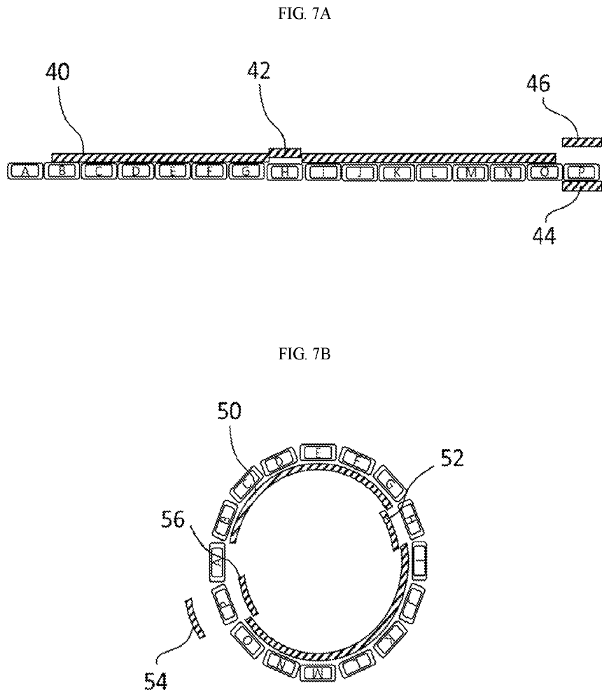

Referring now to FIG. 7 (FIGS. 7A and 7B), exemplary arrays may be seen. Positions A-P may be considered reaction positions used to perform the indicated steps described herein. A reaction vessel may be inserted in a slot (e.g., a slot occurring at each position) and translated along the track. The assay may involve a lateral translation of a sample (e.g., by movement to respective positions along a track) which allow for the indicated reaction position or condition. In some embodiments, a subsequent reaction position may involve no lateral translation of the sample. For example, in FIG. 7A, reaction positions A-P illustrate a series of positions along a linear tract where a sample may be manipulated. In reaction position A, a biological sample and assay reagents including chemiluminescent conjugates and the magnetizable particles having an analyte or a binding partner for an analyte immobilized thereon may be added to a reaction vessel. The reaction vessel may move along the tract to position B where the magnetizable particles begin to be sequestered by magnet 40 as shown, for example, in FIG. 1A. Translation through positions B-E may each involve sequestration of the magnetizable particles. At some positions, for example at position F, liquid media may be removed and at position G, a wash buffer may be added. Position H, which includes magnet 42 further separated from the reaction vessel, may allow the magnetizable particles to disperse and become resuspended or re-dispersed in the wash buffer. Positions I-N may involve additional wash steps (e.g., at position M). Position N may aspire wash buffer from the reaction vessel. At Position O, the first chemiluminescent reagent (e.g., a triggering reagent such as an acid) may be added to the reaction vessel. At Position P, the sequestration of the magnetizable particles may be influenced by magnet 44 and/or 46 (e.g., depending on their relative location to the reaction vessel, depending on their field strength, combinations thereof). At Position P, the presence of the two magnets may allow for the sample to undergo one or more particle movement positions. For example, movement from Position O to P may induce a first particle movement. Manipulation of relative locations of magnets 44 and 46 may induce one or more particle movement reaction position as well without any translational movement of the reaction vessel. In particular embodiments, any particle movement reaction position may involve re-accumulation of the magnetizable particles in the reaction vessel. Following the particle migration steps, the reaction vessel in Position P may be moved (e.g., with a translation arm) into a luminometer where the second chemiluminescent reagent is added and the chemiluminescence is measured. In various implementations, the liquid media is aspirated from the reaction vessel in Position P and added to a new reaction vessel already placed or to be placed in a luminometer for chemiluminescence measurement. FIG. 7B provides a similar embodiment of track for potential reaction positions in a circular carousel or ring format where as a sample is moved around the ring it is exposed to magnets 50, 52, 54, and 56. Following a reaction vessel moving through positions A-P, chemiluminescence may be finally induced through the addition of the second chemiluminescent reagent to the appropriate medium. Addition may occur proximal to photomultiplier tube for light collection such as in a luminometer. The steps described herein may be created by multiple tracts, such as adjacent carousels each providing any indicated reaction position. In various implementations, the systems include a diverter to move a reaction vessel between adjacent carousels.

Referring now to FIG. 1I, a block diagram of a computer system 1100 that can be used in the operations of embodiments of the present disclosure is shown. The system 1100 includes a processor 1110, a memory 1120, a storage device 1130 and an input/output device 1140. Each of the components 1110, 1120, 1130 and 1140 are interconnected using a system bus 1150. The system may include analyzing equipment 1160 for determining a level of one or more analytes of the present disclosure such as one or more biomarkers in a sample.

In embodiments, the processor 1110 is capable of processing instructions for execution within the system 1100. In one embodiment, the processor 1110 is a single-threaded processor. In another embodiment, the processor 1110 is a multi-threaded processor. The processor 1110 is capable of processing instructions stored in the memory 1120 or on the storage device 1130, including for receiving or sending information through the input/output device 1140.

In embodiments, the memory 1120 stores information within the system 1100. In one embodiment, the memory 1120 is a computer-readable medium. In one embodiment, the memory 1120 is a volatile memory unit. In another embodiment, the memory 1120 is a non-volatile memory unit.

In embodiments, the storage device 1130 is capable of providing mass storage for the system 1100. In one embodiment, the storage device 1130 is a computer-readable medium or includes non-transient computer readable medium in accordance with the present disclosure.

In embodiments, the input/output device 1140 provides input/output operations for the system 1100. In one embodiment, the input/output device 1140 includes a keyboard and/or pointing device. In one embodiment, the input/output device 1140 includes a display unit for displaying graphical user interfaces.

The system 1100 can be used to build a database. In embodiments, a method of the present disclosure is performed in a system 1100 disposed within a chemical analyzer. For example, a computer program product can include instructions that cause a processor 1110 to perform the steps of a method of the present disclosure.

Additionally, non-transitory computer readable media containing executable instructions that when executed cause a processor to perform operations including a method as provided herein are provided. For example, a non-transitory computer readable medium containing executable instructions that when executed cause a processor to perform operations including a method as described herein. In embodiments, a non-transitory computer readable medium includes a hard drive, external hard drive, discs, CDs, DVDs, and the like that stores data. In embodiments, software disposed within a physical medium is suitable for use herein.

In some embodiments, a non-transitory computer readable media containing executable instructions that when executed cause a processor to perform operations including a method including process sequences such as: a) adding a first chemiluminescent reagent (e.g., a triggering reagent such as an acid) to a reaction vessel containing magnetizable particles (e.g., paramagnetic particles); and b) moving the magnetizable particles to a different location in the reaction vessel through the first chemiluminescent reagent (as measured, for example, with respect to the container of a reaction vessel).

In embodiments, the present disclosure includes one or more articles of manufacture, such as a system or component thereof including a non-transitory computer-readable medium with instructions encoded thereon, the instructions configured to cause one or more processors to perform a method of Embodiments 1-19 as set forth below.

In some embodiments, a non-transitory computer readable media containing executable instructions that when executed cause a processor to perform operations including one or more of Embodiments 1-19 as set forth below.

### EMBODIMENTS

Embodiment 1: A method embodiment including: a) adding a first chemiluminescent reagent (e.g., a triggering reagent such as an acid) to a reaction vessel containing magnetizable particles (e.g., paramagnetic particles); and b) moving the magnetizable particles to a different location in the reaction vessel through the first chemiluminescent reagent (as measured, for example, with respect to the container of a reaction vessel).

Embodiment 2. The method according to Embodiment 1, wherein the magnetizable particles are moved by application of a magnetic field to the reaction vessel.

Embodiment 3. The method according to Embodiment 1 or Embodiment 2, wherein the magnetizable particles are sequestered in the reaction vessel during or after the addition step by a magnetic field to the reaction vessel.

Embodiment 4. The method according to any one of Embodiments 1-3, wherein the magnetizable particles are not sequestered in the reaction vessel during the addition step (e.g., the particles become suspended in the first chemiluminescent reagent, no magnetic field is applied to the reaction vessel).

Embodiment 5. The method according to any one of Embodiments 1-4, further including: c) separating liquid media from the magnetizable particles after moving the magnetizable particles; and d) adding a second chemiluminescent reagent to the separated liquid media (e.g., in another reaction vessel) or to the separated magnetizable particles.

Embodiment 6. The method according to any one of Embodiments 1-4, further including: c) adding a second chemiluminescent reagent to the magnetizable particles and, optionally, liquid media present (e.g., the first chemiluminescent reagent) after moving the magnetizable particles.

Embodiment 7. The method according to Embodiment 6, wherein the magnetizable particles are clustered in the reaction vessel during the second chemiluminescent reagent addition (e.g., the reaction vessel has a magnet proximal thereto to induce clustering or accumulation).

Embodiment 8. The method according to Embodiment 7, wherein the reaction vessel during the second chemiluminescent reagent addition is symmetric about a major longitudinal axis (e.g., cylindrical such as a cuvette or tube) and the second chemiluminescent reagent is added at position other than the major longitudinal axis.

Embodiment 9. The method according to any one of Embodiments 1-8, wherein the sample is contained in a reaction vessel (e.g., a cuvette, a tube) during addition of the first chemiluminescent reagent, and the reaction vessel has a magnet adjacent thereto during addition of the first chemiluminescent reagent addition position (e.g., to sequester the magnetizable particles along an internal wall of the reaction vessel).

Embodiment 10. The method according to Embodiment 9, wherein moving the magnetizable particles occurs by movement of the reaction vessel (e.g., rotating by, for example, 170-190° or 180°) with respect to the magnet.

Embodiment 11. The method according to Embodiments 9 or 10, wherein moving the magnetizable particles occurs by moving the reaction vessel into a different magnetic field created by a different set of magnets.

Embodiment 12. The method according to any one of Embodiments 1-11, wherein moving the magnetizable particles occurs by moving a magnet in relation to the sample (e.g., as contained in a reaction vessel) and/or modulating (e.g., increasing, decreasing) the magnetic field produced from one or more magnets (e.g., alteration of electrical parameters such as voltage or current to an electromagnet) proximal to the sample.

Embodiment 13. The method according to any one of Embodiments 1-12, wherein the first chemiluminescent reagent is acidic (e.g., a reagent including an acid such as hydrogen peroxide, nitric acid, or a combination thereof) and optionally includes a detergent (e.g., a cationic detergent such as quaternary nitrogen or phosphorus-based salts).

Embodiment 14. The method according to any one of Embodiments 1-13, wherein the second chemiluminescent reagent is basic (e.g., a reagent including a base such as an alkali hydroxide (e.g., sodium hydroxide)) and optionally comprises a detergent (e.g., a cationic detergent such as quaternary nitrogen or phosphorus-based salts).

Embodiment 15. The method according to any one of Embodiments 1-14, further including forming the sample by mixing magnetizable particles having a molecule capable of forming a binding complex with an analyte of interest or binding partner thereof immobilized thereon with a biological sample (e.g., blood serum, urine) and with an assay reagent comprising chemiluminescent conjugates (e.g., acridinium compounds such as acridinium esters or acridinium sulfonamides) capable of forming a binding complex with the molecule immobilized on the magnetizable particles; and optionally, incubating biological sample, the magnetizable particles, and the chemiluminescent conjugates; sequestering the magnetizable particles in a reaction vessel by application of a magnetic field to the reaction vessel; adding a wash buffer (e.g., a buffer solution which may contain one or more salts such as sodium chloride and sodium azide, detergents such as cationic detergents, buffering agents such as phosphate, blocking agents such as bovine serum albumin (BSA) or combinations thereof) to the magnetizable particles is added to the magnetizable particles; and separating liquid media (e.g., from the biological sample, from the assay reagents, from the first chemiluminescent reagent, from the second chemiluminescent reagent, from the wash buffer, from combinations thereof) from the magnetizable particles (e.g., the sequestered magnetizable particles).

Embodiment 16. The method according to Embodiment 15, wherein the wash buffer is added when the magnetizable particles are dispersed throughout the liquid media.

Embodiment 17. The method according to Embodiment 15, wherein the wash buffer is added when the magnetizable particles are sequestered (e.g., through application of a magnetic field to a reaction vessel).

Embodiment 18. The method according to any one of Embodiments 15-17, wherein the method includes at least two wash buffer additions including a first wash buffer addition where the magnetizable particles are dispersed throughout the liquid media as the wash buffer is added; and a second wash buffer addition where the magnetizable particles are sequestered during addition of the wash buffer.

Embodiment 19. The method according to any one of Embodiments 1-18, wherein the method further includes measuring the chemiluminescent light output following addition of the second chemiluminescent reagent (e.g., with a photomultiplier tube such as a luminometer).

Embodiment 20. A system for inducing a chemiluminescent reaction from a chemiluminescent sample comprising magnetizable particles (e.g., paramagnetic particles), wherein the system comprises: an array of consecutive reaction positions for the chemiluminescent sample such that the chemiluminescent sample (or media derived therefrom) can be placed in each reaction position sequentially (e.g., by movement of a reaction vessel (e.g., a cuvette, a tube) comprising a material such as the magnetizable particles and/or liquid media between each reaction position, by aspiration of liquid media in a first reaction vessel and deposition into a second reaction vessel); said array of consecutive reaction positions comprising: a first chemiluminescent reagent addition position where a first chemiluminescent reagent can be added to the chemiluminescent sample; one or more (e.g., from one to ten, from one to five, two, three, four, five, six, seven, eight, nine, ten) particle movement positions wherein a magnetic field different from a previous position (e.g., the first chemiluminescent regent addition position, the previous particle movement position) can be applied to the chemiluminescent sample to induce movement of magnetizable particles in the first chemiluminescent reagent; a second chemiluminescent reagent addition position where a second chemiluminescent reagent is added to the chemiluminescent sample (or portion thereof or derived therefrom) after the one or more particle movement positions to induce a chemiluminescent reaction from the chemiluminescent sample (or portion thereof or derived therefrom).

Embodiment 21. The system according to Embodiment 20, wherein the array comprises a liquid transfer position where a liquid chemiluminescent sample is separated from the magnetizable particles (e.g., by aspiration with a pipette) following the one or more particle movement positions; and the second chemiluminescent reagent vessel position adds the second chemiluminescent reagent to the separated liquid sample (e.g., in a different reaction vessel such as a cuvette or tube from the reaction vessel used in the first chemiluminescent position).

Embodiment 22. The system according to Embodiment 20, wherein the second chemiluminescent reagent is added to the chemiluminescent sample comprising the magnetizable particles (e.g., in the same reaction vessel that the first chemiluminescent sample was added such as a cuvette or tube).

Embodiment 23. The system according to Embodiment 22, wherein the magnetizable particles are clustered in the reaction vessel at the second chemiluminescent reagent addition position (e.g., the reaction vessel of the second chemiluminescent position has a magnet proximal thereto to induce clustering).

Embodiment 24. The system according to Embodiment 23, wherein the reaction vessel in the second chemiluminescent position is symmetric about a major longitudinal axis (e.g., cylindrical such as a cuvette or tube) and the second chemiluminescent reagent is added at position other than the major longitudinal axis.

Embodiment 25. The system according to any one of Embodiments 20-24, wherein the chemiluminescent sample is contained in a reaction vessel (e.g., a cuvette, a tube) in the first chemiluminescent reagent addition position and the first chemiluminescent reagent is added to the reaction vessel.

Embodiment 26. The system according to Embodiment 25, wherein the reaction vessel has a magnet adjacent thereto during the first chemiluminescent reagent addition position (e.g., to sequester the magnetizable particles along an internal wall of the reaction vessel).

Embodiment 27. The system according to Embodiments 25 or 26, wherein at least one of the different magnetic fields in at least one particle movement position is created on the chemiluminescent sample by rotating the reaction vessel (e.g., rotating by, for example, 170-190° or 180°).

Embodiment 28. The system according to any one of Embodiments 25-27, wherein at least one of the different magnetic fields in at least one particle movement position is created by moving the reaction vessel into a different magnetic field created by a different set of magnets.

Embodiment 29. The system according to any one of Embodiments 20-28, wherein at least one of the different magnetic fields in at least one particle movement position is created by moving a magnet in relation to the sample (e.g., as contained in a reaction vessel) and/or modulating (e.g., increasing, decreasing) the magnetic field produced from one or more magnets (e.g., alteration of electrical parameters such as voltage or current to an electromagnet) proximal to the sample.

Embodiment 30. The system according to any one of Embodiments 20-29, wherein the first chemiluminescent reagent is acidic (e.g., a reagent comprising an acid such as hydrogen peroxide, nitric acid, or a combination thereof) and optionally includes a detergent (e.g., a cationic detergent such as quaternary nitrogen or phosphorus-based salts).

Embodiment 31. The system according to any one of Embodiments 20-30, wherein the second chemiluminescent reagent is basic (e.g., a reagent comprising a base such as an alkali hydroxide (e.g., sodium hydroxide)) and optionally comprises a detergent (e.g., a cationic detergent such as quaternary nitrogen or phosphorus-based salts).

Embodiment 32. The system according to any one of Embodiments 20-31, wherein the system further comprises reaction positions to form the chemiluminescent sample from a biological sample, wherein the reaction positions to form the chemiluminescent sample comprising of at least one reaction position selected from: a biological sample addition position where the biological sample is added to a reaction vessel; an assay reagent position where assay reagents (e.g., magnetizable particles having a molecule capable of forming a binding complex with an analyte of interest or binding partner thereof immobilized thereon, chemiluminescent conjugates such as acridinium compounds including acridinium esters and acridinium sulfonamides capable of forming a binding complex with the molecule immobilized on the magnetizable particles, or both) added to a reaction vessel (e.g., the reaction vessel containing the biological sample); an incubation position for competitively binding analytes from the biological sample and the chemiluminescent compounds to the magnetizable particles; a sequester position wherein the magnetizable particles are magnetically sequestered from the liquid media of the biological sample and assay reagents; a separation position where liquid media (e.g., from the biological sample, from the assay reagents) is separated from the magnetizable particles (e.g., the sequestered magnetizable particles); a wash position where a wash buffer (e.g., a buffer solution which may contain one or more salts such as sodium chloride and sodium azide, detergents such as cationic detergents, buffering agents such as phosphate, blocking agents such as bovine serum albumin (BSA) or combinations thereof) is added to the magnetizable particles; and a wash buffer aspiration position where wash buffer is separated from the magnetizable particles to form the chemiluminescent sample.

Embodiment 33. The system according to Embodiment 32, wherein the system comprises a wash position where the magnetizable particles are dispersed throughout the liquid media as the wash buffer is added.

Embodiment 34. The system according to Embodiment 32, wherein the system comprises a wash position where the magnetizable particles are sequestered during addition of the wash buffer.

Embodiment 35. The system according to any one of Embodiment 32-34, wherein the system comprises at least two wash positions including a first wash position where the magnetizable particles are dispersed throughout the liquid media as the wash buffer is added; and a second wash position where the magnetizable particles are sequestered during addition of the wash buffer.

Embodiment 36. The system according to any one of Embodiment 20-35, wherein the system further comprises a photomultiplier tube (e.g., a luminometer) to measure the chemiluminescent light output following addition of the second chemiluminescent reagent.

Embodiment 37. The system according to any one of Embodiment 20-35, wherein the system further includes non-transient computer readable media for executing a method of the present disclosure including Embodiments 1-19.

### EXAMPLES

The following examples illustrate specific aspects of the instant description. The examples should not be construed as limiting, as the example merely provides specific understanding and practice of the embodiments and its various aspects.

### Example 1

Chemiluminescent beads produced from standards of different concentrations of Troponin I (TnI) were prepared. A similar preparation is disclosed in R. Payne European Heart Journal 38 (2017): ehx502.P2754 which is hereby incorporated by reference and particularly in relation to bead preparation and the Siemens ADVIA Centaur high sensitivity Troponin I sandwich assay. This standard assay has a limit of detection of 0.006 ng/mL, where a biological sample needs to have at least 0.006 ng/mL to measure a detectable concentration difference. Beads were washed with wash buffer and a magnetic field was applied for 60 seconds during wash. The wash buffer was removed and acid was added. Beads were completely suspended in acid and sequestered magnetically. The acid supernatant was transferred to a new cuvette and base added to produce signal. The signal to noise was measured at four different TnI concentrations in repeated experiments. Those results are shown in Table 1 illustrating the (±) 1 standard deviation for the measured S/N.

TABLE 1

| | | | |
|---|---|---|---|
| Sample | Concentration | | |
| Number | (ng/mL) | S/N (bead) | S/N (no bead) |

Experiment 1

| | | | |
|---|---|---|---|
| S0 | 0 | | |
| S1 | 0.024 | 1.43 ± 0.18 | 1.74 ± 0.48 |
| S2 | 0.048 | 2.58 ± 0.45 | 3.29 ± 0.52 |
| S3 | 0.091 | 5.38 ± 0.82 | 6.12 ± 0.78 |
| S4 | 0.484 | 26.83 ± 3.13 | 33.02 ± 4.82 |

Experiment 2

| | | | |
|---|---|---|---|
| S0 | 0 | | |
| S2 | 0.048 | 12.6 ± 1.53 | 13.8 ± 4.27 |
| S4 | 0.484 | 63.7 ± 9.6 | 121.82 ± 38.97 |
| S6 | 5.39 | 454.9 ± 48.33 | 1160.2 ± 365.85 |

As can be seen, an improvement in signal to noise can be seen when beads are removed prior to relative light unit measurement.

Assays were compared to the standard TnI assay (std TnI-Ultra assay-base dispersion in the center) measuring 0 and 0.024 ng/mL concentration of TnI. Table 2 provides the comparison of these results allowing for the comparison of signal to noise for these measurements including alterations to this assay involving magnetic migration and an off-center addition of the second chemiluminescent reagent (base).

TABLE 2

| | | | | | | TnI-Ultra assay with 4× | | |
|---|---|---|---|---|---|---|---|---|
| | | | TnI-Ultra assay with 4× | | | Magnetic Switch, readout | | |
| | Std TnI-Ultra assay | | Magnetic Switch, read-out | | | in SAME cuvette (with beads | | |
| | (Base dispersion | | in NEW cuvette (no beads), | | | sequestered to one side), | | |
| | in center) | | center base dispersion | | | OFF-center base dispersion | | |
| | Concentration (ng/mL) | | | | | | | |
| | 0 | 0.024 | 0 | 0.024 | | 0 | 0.024 | |
| | RLU | RLU | RLU | RLU | | RLU | RLU | |
| | 3692 | 10046 | 1668 | 6309 | | 1411 | 5933 | |
| | 3219 | 8137 | 1053 | 6781 | | 1597 | 6514 | |
| | 2675 | 10684 | 902 | 7187 | | 1518 | 6589 | |
| | | 10751 | 1383 | 7726 | | 1673 | 6512 | |
| | 4096 | 9018 | 1050 | 5256 | | 1689 | 6178 | |
| | | | 1068 | 6935 | | | | |
| | | | 963 | | | | | |
| | | | 1055 | | | | | |
| Average | 3413.8 | 9727.2 | 1142.8 (↓) | 6699.0 | (↓) | 1577.6 (↓) | 6345.2 | (↓) |
| SD | 602.9 | 1128.8 | 254.6 | 847.3 | | 115.3 | 280.0 | |
| % CV | 17.7 | 11.6 | 22.3 | 12.6 | | 7.3 | 4.4 | |
| SNR | | 2.8 | | 5.9 | (↓) | | 4.0 | (↓) |

| | | | |
|---|---|---|---|
| SNR (with error) | 2.8 ± 0.6 | 5.9 ± 1.5 | 4.0 ± 0.34 |

| | | | | |
|---|---|---|---|---|
| % Reduction from Standard | 66.5 | 31.1 | 53.8 | 34.8 |

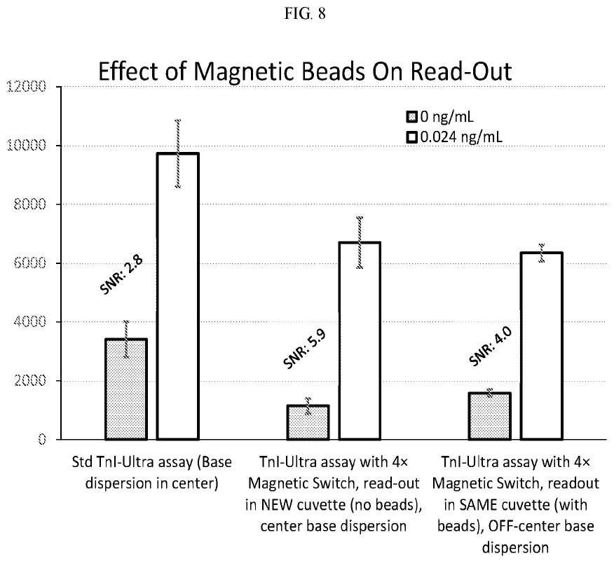

As can be seen, the modifications to the standard assay protocol including particle movement in the first chemiluminescent reagent (acid) resulted in decreases (↓) in RLU measurements for both signal and noise. However, the decrease in noise was more pronounced resulting in an increase (↑) in signal to noise. Furthermore, read-outs in a NEW reaction vessel following magnetic switching yields increased signal to noise as comparison to readings in the same reaction vessel. FIG. 8 provides a comparison of the RLU measured for both noise (left for each protocol) and signal (right for each protocol).

### Example 2

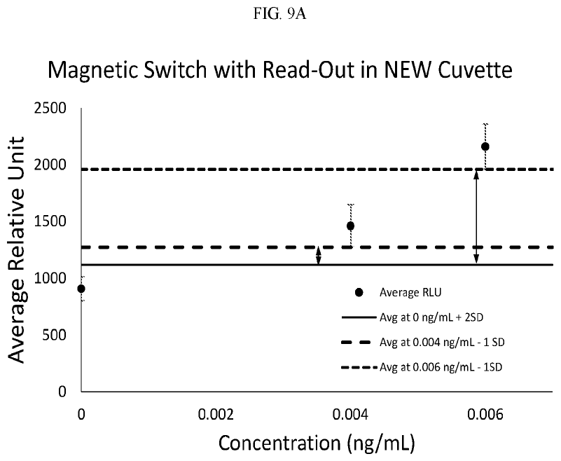

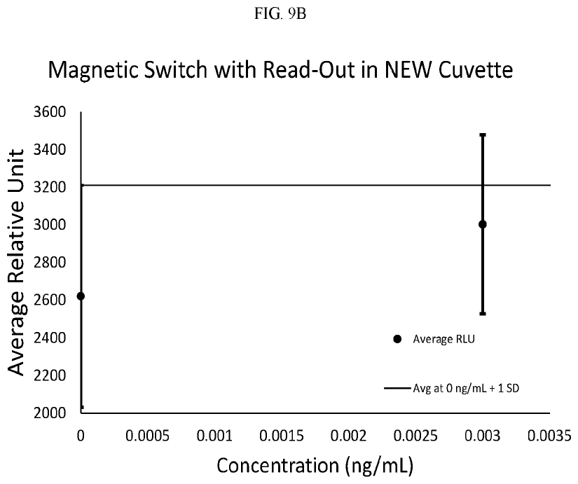

The increased signal to noise of the presently disclosed systems and methods was shown to decrease the limit of detection (LOD) and allow measurements below the standard assay. Standards of TnI were prepared at 0 and 0.003, and 0.006 ng/mL, at or below the limit of detection (LOD) of the standard assay, and assays were run using the standard protocol in a sandwich assay format and a similar protocol involving magnetic switching and particle migration with deposition of the liquid media in a new cuvette. Tables 3 provides the results of this comparison and various statistical parameters of the repeated measurements including the averages (Avg) at each concentration, the measured standard deviation (SD), the coefficient of variability (as a percentage of the average), and the signal to noise (SNR). FIG. 9A plots the average values from the 0, 0.003, and 0.006 ng/mL measurements for the magnetic switch and FIG. 9B plots the average values from these measurements for the standard protocol with statistical values.

TABLE 3

| | Mag Switch method with | | | | |
|---|---|---|---|---|---|
| | read-out in NEW Cuvette | | | Std TnI Method | |
| | Concentration (ng/mL) | | | | |
| | 0 | 0.004 | 0.006 | 0 | 0.003 |
| 1 | 922 | 1605 | 2217 | 2127 | 3033 |
| 2 | 979 | 1277 | 2307 | 1972 | 2726 |
| 3 | 725 | 1324 | 2445 | 3283 | 2584 |
| 4 | 749 | 1452 | 1999 | 3155 | 3800 |
| 5 | 971 | 1736 | 2072 | 2570 | 2874 |
| 6 | 874 | 1548 | 1916 | | |
| 7 | 1012 | 1674 | | | |
| 8 | 934 | 1255 | | | |
| 9 | 1000 | 1273 | | | |
| Avg | 907.33 | 1460.44 | 2159.33 | 2621.40 | 3003.40 |
| SD | 105.52 | 187.01 | 199.64 | 589.75 | 475.67 |
| % CV | 11.63 | 12.81 | 9.25 | 22.50 | 15.84 |
| Avg + 2SD | 1118.38 | | | 3800.91 | |
| Avg − 1SD | | 1273.43 | 1959.69 | | 2527.73 |
| SNR | | 1.61 | 2.38 | | 1.15 |

| | | | |
|---|---|---|---|
| SNR with errors | 1.61 ± | 2.38 ± | 1.15 ± |
| | 0.28 | 0.35 | 0.31 |

Measurements were again performed on TnI standards at 0 and 0.003 ng/mL (below the limit of detection (LOD) of the standard assay), and assays were run using the standard protocol in a sandwich assay as discussed above format and a similar protocol involving magnetic switching and particle migration with deposition of the liquid media in a new cuvette. Table 4 provides the results of those measurements.

TABLE 4

| Mag Switch method with | |
|---|---|
| read-out in NEW Cuvette | Std TnI Method |

Concentration (ng/mL)

| | 0 | 0.004 | 0 | 0.004 |
|---|---|---|---|---|
| 1 | 1080 | 1884 | 2555 | 3340 |
| 2 | 1132 | 1895 | 3048 | 2732 |
| 3 | 966 | 1751 | 3038 | 3071 |
| 4 | 973 | 1818 | 2576 | 3704 |
| 5 | 1042 | 1411 | 3023 | 3490 |
| 6 | 1019 | 1856 | | |
| 7 | 939 | 1814 | | |
| 8 | 956 | 1817 | | |
| 9 | 1023 | 1869 | | |
| 10 | 1043 | 2162 | | |
| Avg | 1017.3 | 1790.56 | 2848.00 | 3267.4 |
| SD | 60.44 | 149.04 | 258.15 | 377.71 |
| % CV | 5.94 | 8.32 | 9.06 | 11.56 |
| Avg + 2SD | 1138.18 | | 3364.29 | |
| Avg − 1SD | | 1641.51 | | 2889.69 |
| SNR | | 1.76 | | 1.15 |

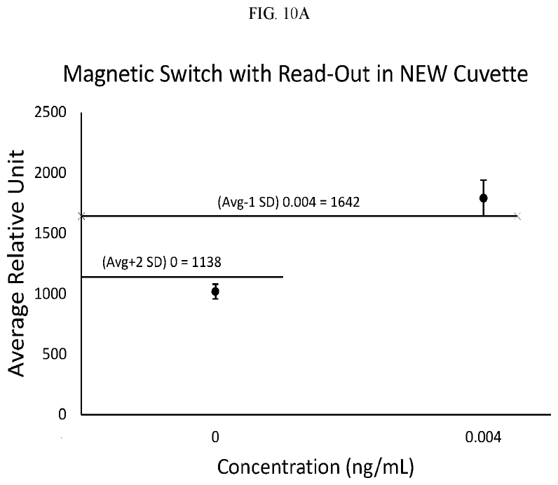

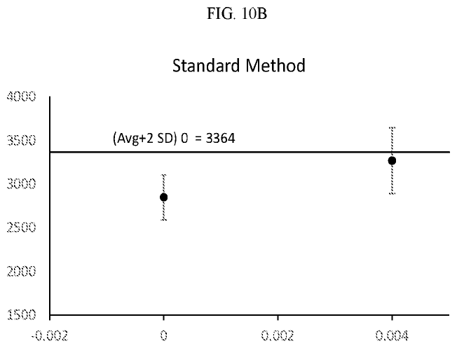

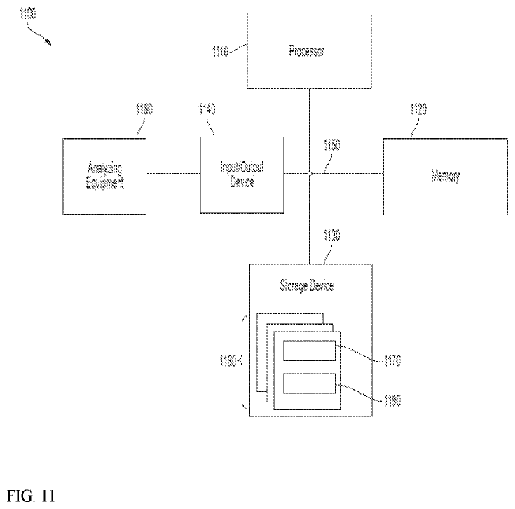

FIG. 10A plots the average values from the 0 and 0.004 ng/mL measurements for the magnetic switch and FIG. 10B plots the average values from these measurements for the standard protocol with statistical values. Error bars represent one standard of deviation. As can be seen, in the standard assay, a concentration of 0.004 cannot be distinguished from 0 concentration. In contrast, using the methods as provided herein, the magnetic switch allows measurement below the LOD of the standard assay. The RLU value is clearly separable for 0 (average+2SD) and 0.004 (average−1SD) when performed using a magnetic switch in an immunoassay.

As various changes can be made in the above-described subject matter without departing from the scope and spirit of the present disclosure, it is intended that all subject matter contained in the above description, or defined in the appended claims, be interpreted as descriptive and illustrative of the present disclosure. Many modifications and variations of the present disclosure are possible in light of the above teachings. Accordingly, the present description is intended to embrace all such alternatives, modifications and variances which fall within the scope of the appended claims.

All documents cited or referenced herein, and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

1. A method comprising:
a) adding a first chemiluminescent reagent (e.g., a triggering reagent such as an acid) to a reaction vessel containing magnetizable particles (e.g., paramagnetic particles); and
b) moving the magnetizable particles to a different location in the reaction vessel through the first chemiluminescent reagent (as measured, for example, with respect to the container of a reaction vessel).

2. The method according to claim 1, wherein the magnetizable particles are moved by application of a magnetic field to the reaction vessel.

3. The method according to claim 1, wherein the magnetizable particles are sequestered in the reaction vessel during or after the addition step by a magnetic field to the reaction vessel.

4. The method according to claim 1, wherein the magnetizable particles are not sequestered in the reaction vessel during the addition step (e.g., the particles become suspended in the first chemiluminescent reagent, no magnetic field is applied to the reaction vessel).

5. The method according to claim 1, further comprising:
c) separating liquid media from the magnetizable particles after moving the magnetizable particles; and
d) adding a second chemiluminescent reagent to the separated liquid media (e.g., in another reaction vessel) or to the separated magnetizable particles.

6. The method according to claim 1, further comprising:
c) adding a second chemiluminescent reagent to the magnetizable particles and, optionally, liquid media present (e.g., the first chemiluminescent reagent) after moving the magnetizable particles.

7. The method according to claim 6, wherein the magnetizable particles are clustered in the reaction vessel during the second chemiluminescent reagent addition (e.g., the reaction vessel has a magnet proximal thereto to induce clustering or accumulation).

8. The method according to claim 7, wherein the reaction vessel during the second chemiluminescent reagent addition is symmetric about a major longitudinal axis (e.g., cylindrical such as a cuvette or tube) and the second chemiluminescent reagent is added at position other than the major longitudinal axis.

9. The method according to claim 1, wherein the sample is contained in a reaction vessel (e.g., a cuvette, a tube) during addition of the first chemiluminescent reagent, and the reaction vessel has a magnet adjacent thereto during addition of the first chemiluminescent reagent addition position (e.g., to sequester the magnetizable particles along an internal wall of the reaction vessel).

10. The method according to 9, wherein moving the magnetizable particles occurs by movement of the reaction vessel (e.g., rotating by, for example, 170-190° or 180°) with respect to the magnet.

11. The method according to claim 9, wherein moving the magnetizable particles occurs by moving the reaction vessel into a different magnetic field created by a different set of magnets.

12. The method according to claim 1, wherein moving the magnetizable particles occurs by moving a magnet in relation to the sample (e.g., as contained in a reaction vessel) and/or modulating (e.g., increasing, decreasing) the magnetic field produced from one or more magnets (e.g., alteration of electrical parameters such as voltage or current to an electromagnet) proximal to the sample.

13. The method according to claim 1, wherein the first chemiluminescent reagent is acidic (e.g., a reagent comprising an acid such as hydrogen peroxide, nitric acid, or a combination thereof) and optionally includes a detergent (e.g., a cationic detergent such as quaternary nitrogen or phosphorus-based salts).

14. The method according to claim 1, wherein the second chemiluminescent reagent is basic (e.g., a reagent comprising a base such as an alkali hydroxide (e.g., sodium hydroxide)) and optionally comprises a detergent (e.g., a cationic detergent such as quaternary nitrogen or phosphorus-based salts).

15. The method according to claim 1, further comprising forming the sample by mixing magnetizable particles having a molecule capable of forming a binding complex with an analyte of interest or binding partner thereof immobilized thereon with a biological sample (e.g., blood serum, urine) and with an assay reagent comprising chemiluminescent conjugates (e.g., acridinium compounds such as acridinium esters or acridinium sulfonamides) capable of forming a binding complex with the molecule immobilized on the magnetizable particles; and optionally,
incubating biological sample, the magnetizable particles, and the chemiluminescent conjugates;
sequestering the magnetizable particles in a reaction vessel by application of a magnetic field to the reaction vessel;
adding a wash buffer (e.g., a buffer solution which may contain one or more salts such as sodium chloride and sodium azide, detergents such as cationic detergents, buffering agents such as phosphate, blocking agents such as bovine serum albumin (BSA) or combinations thereof) to the magnetizable particles is added to the magnetizable particles; and
separating liquid media (e.g., from the biological sample, from the assay reagents, from the first chemiluminescent reagent, from the second chemiluminescent reagent, from the wash buffer, from combinations thereof) from the magnetizable particles (e.g., the sequestered magnetizable particles).

16. The method according to claim 15, wherein the wash buffer is added when the magnetizable particles are dispersed throughout the liquid media.

17. The method according to claim 15, wherein the wash buffer is added when the magnetizable particles are sequestered (e.g., through application of a magnetic field to a reaction vessel).

18. The method according to claim 15, wherein the method comprises at least two wash buffer additions including a first wash buffer addition where the magnetizable particles are dispersed throughout the liquid media as the wash buffer is added; and a second wash buffer addition where the magnetizable particles are sequestered during addition of the wash buffer.

19. The method according to claim 1, wherein the method further comprises measuring the chemiluminescent light output following addition of the second chemiluminescent reagent (e.g., with a photomultiplier tube such as a luminometer).

20. A system for inducing a chemiluminescent reaction from a chemiluminescent sample comprising magnetizable particles (e.g., paramagnetic particles), wherein the system comprises:
an array of consecutive reaction positions for the chemiluminescent sample such that the chemiluminescent sample (or media derived therefrom) can be placed in each reaction position sequentially (e.g., by movement of a reaction vessel (e.g., a cuvette, a tube) comprising a material such as the magnetizable particles and/or liquid media between each reaction position, by aspiration of liquid media in a first reaction vessel and deposition into a second reaction vessel); said array of consecutive reaction positions comprising:
a first chemiluminescent reagent addition position where a first chemiluminescent reagent can be added to the chemiluminescent sample;
one or more (e.g., from one to ten, from one to five, two, three, four, five, six, seven, eight, nine, ten) particle movement positions wherein a magnetic field different from a previous position (e.g., the first chemiluminescent regent addition position, the previous particle movement position) can be applied to the chemiluminescent sample to induce movement of magnetizable particles in the first chemiluminescent reagent;
a second chemiluminescent reagent addition position where a second chemiluminescent reagent is added to the chemiluminescent sample (or portion thereof or derived therefrom) after the one or more particle movement positions to induce a chemiluminescent reaction from the chemiluminescent sample (or portion thereof or derived therefrom).

21. The system according to claim 20, wherein the array comprises a liquid transfer position where a liquid chemiluminescent sample is separated from the magnetizable particles (e.g., by aspiration with a pipette) following the one or more particle movement positions; and the second chemiluminescent reagent vessel position adds the second chemiluminescent reagent to the separated liquid sample (e.g., in a different reaction vessel such as a cuvette or tube from the reaction vessel used in the first chemiluminescent position).

22. The system according to claim 20, wherein the second chemiluminescent reagent is added to the chemiluminescent sample comprising the magnetizable particles (e.g., in the same reaction vessel that the first chemiluminescent sample was added such as a cuvette or tube).

23. The system according to claim 22, wherein the magnetizable particles are clustered in the reaction vessel at the second chemiluminescent reagent addition position (e.g., the reaction vessel of the second chemiluminescent position has a magnet proximal thereto to induce clustering).

24. The system according to claim 23, wherein the reaction vessel in the second chemiluminescent position is symmetric about a major longitudinal axis (e.g., cylindrical such as a cuvette or tube) and the second chemiluminescent reagent is added at position other than the major longitudinal axis.

25. The system according to claim 20, wherein the chemiluminescent sample is contained in a reaction vessel (e.g., a cuvette, a tube) in the first chemiluminescent reagent addition position and the first chemiluminescent reagent is added to the reaction vessel.

26. The system according to claim 25, wherein the reaction vessel has a magnet adjacent thereto during the first chemiluminescent reagent addition position (e.g., to sequester the magnetizable particles along an internal wall of the reaction vessel).

27. The system according to claim 25, wherein at least one of the different magnetic fields in at least one particle movement position is created on the chemiluminescent sample by rotating the reaction vessel (e.g., rotating by, for example, 170-190° or 180°).

28. The system according to claim 25, wherein at least one of the different magnetic fields in at least one particle movement position is created by moving the reaction vessel into a different magnetic field created by a different set of magnets.

29. The system according to claim 20, wherein at least one of the different magnetic fields in at least one particle movement position is created by moving a magnet in relation to the sample (e.g., as contained in a reaction vessel) and/or modulating (e.g., increasing, decreasing) the magnetic field produced from one or more magnets (e.g., alteration of electrical parameters such as voltage or current to an electromagnet) proximal to the sample.

30. The system according to claim 20, wherein the first chemiluminescent reagent is acidic (e.g., a reagent comprising an acid such as hydrogen peroxide, nitric acid, or a combination thereof) and optionally includes a detergent (e.g., a cationic detergent such as quaternary nitrogen or phosphorus-based salts).

31. The system according to claim 20, wherein the second chemiluminescent reagent is basic (e.g., a reagent comprising a base such as an alkali hydroxide (e.g., sodium hydroxide)) and optionally comprises a detergent (e.g., a cationic detergent such as quaternary nitrogen or phosphorus-based salts).

32. The system according to claim 20, wherein the system further comprises reaction positions to form the chemiluminescent sample from a biological sample, wherein the reaction positions to form the chemiluminescent sample comprising of at least one reaction position selected from:
a biological sample addition position where the biological sample is added to a reaction vessel;
an assay reagent position where assay reagents (e.g., magnetizable particles having a molecule capable of forming a binding complex with an analyte of interest or binding partner thereof immobilized thereon, chemiluminescent conjugates such as acridinium compounds including acridinium esters and acridinium sulfonamides capable of forming a binding complex with the molecule immobilized on the magnetizable particles, or both) added to a reaction vessel (e.g., the reaction vessel containing the biological sample);
an incubation position for competitively binding analytes from the biological sample and the chemiluminescent compounds to the magnetizable particles;
a sequester position wherein the magnetizable particles are magnetically sequestered from the liquid media of the biological sample and assay reagents;
a separation position where liquid media (e.g., from the biological sample, from the assay reagents) is separated from the magnetizable particles (e.g., the sequestered magnetizable particles);
a wash position where a wash buffer (e.g., a buffer solution which may contain one or more salts such as sodium chloride and sodium azide, detergents such as cationic detergents, buffering agents such as phosphate, blocking agents such as bovine serum albumin (BSA) or combinations thereof) is added to the magnetizable particles; and
a wash buffer aspiration position where wash buffer is separated from the magnetizable particles to form the chemiluminescent sample.

33. The system according to claim 32, wherein the system comprises a wash position where the magnetizable particles are dispersed throughout the liquid media as the wash buffer is added.

34. The system according to claim 32, wherein the system comprises a wash position where the magnetizable particles are sequestered during addition of the wash buffer.

35. The system according to claim 32, wherein the system comprises at least two wash positions including a first wash position where the magnetizable particles are dispersed throughout the liquid media as the wash buffer is added; and a second wash position where the magnetizable particles are sequestered during addition of the wash buffer.

36. The system according to claim 20, wherein the system further comprises a photomultiplier tube (e.g., a luminometer) to measure the chemiluminescent light output following addition of the second chemiluminescent reagent.
